(19) United States
(12) Patent Application Publication
(10) Pub. No.: US 2026/0198794 A1
(43) Pub. Date: Jul. 16, 2026

(54) BIODEGRADABLE SENSOR FOR PRESSURE MEASUREMENT AND METHOD FOR MANUFACTURING SAME

(71) Applicants: KOREA UNIVERSITY RESEARCH AND BUSINESS FOUNDATION, Seoul (KR); SEOUL NATIONAL UNIVERSITY HOSPITAL, Seoul (KR)

(72) Inventors: Ja-Hyun KOO, Seoul (KR); Won-Sang CHO, Seoul (KR); Min-Ki HONG, Seoul (KR); Seung-Hun HAN, Seoul (KR)

(73) Assignees: KOREA UNIVERSITY RESEARCH AND BUSINESS FOUNDATION, Seoul (KR); SEOUL NATIONAL UNIVERSITY HOSPITAL, Seoul (KR)

(21) Appl. No.: 19/137,137

(22) PCT Filed: Dec. 1, 2023
(86) PCT No.: PCT/KR2023/095097

§ 371 Date: Jun. 9, 2025

(30) Foreign Application Priority Data
Dec. 9, 2022 (KR) 10-2022-0171086
Oct. 27, 2023 (KR) 10-2023-0145461

(51) Int. Cl. A61B 5/03; A61B 5/00; A61L 31/02; A61L 31/06; A61L 31/14

(52) U.S. Cl. CPC A61B 5/031; A61B 5/6868; A61L 31/022; A61L 31/06; A61L 31/148; A61B 2562/0247; A61B 2562/125; A61B 2562/166

(57) ABSTRACT

The present invention provides a biodegradable sensor for pressure measurement, comprising: a first electrode including a first metal degradable in vivo; a second electrode including a second metal degradable in vivo; and a dielectric portion disposed between the first and second electrodes and including a polymer degradable in vivo, wherein the sensor is capable of measuring pressure based on a variation in spacing between the first and second electrodes.

### TECHNICAL FIELD

The present invention relates to a biodegradable sensor for pressure measurement and a method for manufacturing the same.

### BACKGROUND ART

The present disclosure relates to a biodegradable sensor for measuring intracranial pressure (ICP), and more particularly, to a biodegradable pressure sensor capable of being implanted in the body to measure intracranial pressure in real time, which is safely decomposed and absorbed by surrounding tissues after its intended use.

For patients suffering from cerebrovascular and cardiovascular disorders, continuous assessment of their physiological status is a critical factor in ensuring accurate diagnosis and effective treatment. In hospital settings, parameters such as blood pressure, heart rate, oxygen saturation, and blood test results are frequently monitored.

Unlike other parts of the body, the central nervous system—including the brain and spinal cord—is enclosed within a unique protective structure formed by the skull and the meninges. This anatomical structure significantly limits the accessibility for direct physiological monitoring. Moreover, blood vessels supplying oxygen and nutrients to the central nervous system possess an intrinsic autoregulatory mechanism, enabling the maintenance of constant cerebral blood flow (CBF) and oxygen partial pressure despite fluctuations in systemic circulation. However, to date, no precise and noninvasive method exists for directly measuring such parameters.

Conventionally, cerebral perfusion pressure (CPP) has been estimated indirectly using measured values of intracranial pressure (ICP) and mean arterial blood pressure (MAP), based on the formula:

$$CPP = MAP - ICP .$$

Treatment strategies have been guided by this calculated CPP value in an effort to maintain appropriate CBF.

Due to the importance of accurate ICP monitoring, there is a critical demand for technologies that enable real-time, continuous, and minimally invasive measurement of ICP, ideally without breaching the protective skull and meninges.

However, current methods require surgical incision of the scalp, drilling through the skull, and penetrating the dura mater to insert a pressure sensor into the brain parenchyma or ventricular system. During this period—typically one to two weeks—the implanted sensor remains connected to external monitoring systems via wired interfaces. This procedure poses substantial risks, including potential brain injury during implantation, as well as postoperative complications such as infection, pain, hemorrhage, and neurological deficits. Additionally, the overall treatment cost and risk of surgical sequelae add further burden to both patients and medical systems.

To address these challenges, the present invention proposes a biodegradable implantable medical device that allows for safe, real-time ICP monitoring and is capable of being naturally degraded and absorbed by the body after completing its functional lifespan.

Specifically, the invention relates to a biodegradable intracranial pressure sensor that is inserted into deep brain regions, performs real-time pressure sensing, and undergoes hydrolytic degradation, thereby being safely absorbed into surrounding biological tissues without the need for removal.

### DETAILED DESCRIPTION

### Problems to be Solved

It is an object of the present invention to provide a biodegradable sensor that can be implanted in the body to continuously monitor pressure and is capable of undergoing biodegradation after a certain period, thereby eliminating the need for a secondary surgical removal procedure.

Another object of the present invention is to provide a method for manufacturing the biodegradable sensor as described above.

Still another object of the present invention is to provide a composite sensor assembly comprising a plurality of biodegradable sensors, which reduces signal noise and enhances pressure measurement accuracy.

### Means to Solve the Problems

According to one aspect of the present invention, a biodegradable sensor for pressure measurement comprises: a first electrode comprising a first metal that is biodegradable in vivo; a second electrode comprising a second metal that is biodegradable in vivo; and a dielectric portion disposed between the first and second electrodes, the dielectric portion comprising a polymer that is biodegradable in vivo, wherein pressure is measured based on a change in distance between the first and second electrodes.

In one embodiment, the biodegradable sensor may be configured to measure pressure using a capacitive sensing mechanism.

In one embodiment, the dielectric portion may take one of the following forms: a bulk form filled internally, a frame form having an internal cavity, or a protruded form with raised structures.

In one embodiment, the bulk form may be implemented as a thin film having a thickness ranging from 50 nm to 200 μm.

In one embodiment, the frame form with an internal cavity includes a dielectric frame having: a first surface facing the first electrode; a second surface facing the second electrode and spaced apart from the first surface across the cavity; and a third surface connecting the edges of the first and second surfaces, forming the side walls, wherein the thickness of the dielectric frame may be 10 μm to 150 μm, and the spacing between the first and second surfaces may be 50 μm to 200 μm.

In another embodiment, the protruded form may comprise circular or polygonal pillars disposed on a bottom surface, wherein the protrusions are spaced at regular intervals.

The spacing between the base areas of the protrusions may range from 10 μm to 500 μm, and the height of the protrusions may range from 100 μm to 500 μm.

The dielectric constant of the dielectric portion may range from 0.1 pF/m to 0.5 pF/m.

The first and second electrodes may comprise at least one of magnesium (Mg), zinc (Zn), tungsten (W), and molybdenum (Mo).

The polymer forming the dielectric may comprise at least one of: poly(lactic-co-glycolic acid) (PLGA), poly(butanedithiol pentenoic anhydride) (PBTPA), polyglycolic acid (PGA), polycaprolactone (PCL), poly(glycerol sebacate) (PGS), polylactic acid (PLA), gelatin methacryloyl (GelMA), gelatin methacryloyl-polyethylene glycol diacrylate (GelMA-PEGDA), and polyurethane (PU). In one embodiment, the polymer dielectric is PBTPA, and may include PLGA:PBTPA:PGA in a molar ratio of 1:1:2.5 to 1:4:7.

According to another aspect of the present invention, a method for manufacturing a biodegradable sensor having at least one of the features described above may comprise: forming a first electrode by applying a paste containing the first metal or attaching a film comprising the first metal; forming a dielectric portion over the first electrode by applying a biodegradable polymer dielectric; and forming a second electrode over the dielectric portion by applying a paste containing the second metal or attaching a film comprising the second metal.

In one embodiment, when the dielectric portion is of bulk form, it may be formed by screen printing or molding the polymer dielectric.

In another embodiment, when the dielectric portion is of frame form with an internal cavity, forming the dielectric portion may comprise: forming a first surface by applying the polymer dielectric over the first electrode; forming a third surface by applying the polymer dielectric on the edges of the first surface to define a cavity; and forming a second surface over the third surface using the polymer dielectric to face the second electrode.

In yet another embodiment, if the dielectric portion is of protruded form, the method may comprise molding the polymer into the protruded structure and disposing it over the first electrode.

In one embodiment, the polymer dielectric is PBTPA and may include pentenoic anhydride, triallyl triazinetrione, and butanedithiol in a ratio of 1:1:2.5 to 1:4:7.

The method may further include repeating the steps of forming the dielectric portion and the second electrode after the second electrode is initially formed, thereby constructing a multilayer structure.

According to another aspect of the present invention, a composite sensor assembly may comprise: a sensing unit, which includes a biodegradable sensor having at least one of the features described above, and is configured to generate a variable electrical signal in response to changes in external pressure; and a reference unit comprising a third electrode and configured to generate a constant electrical signal regardless of external pressure changes.

The composite sensor assembly detects external pressure variations by comparing the electrical signal from the sensing unit with that of the reference unit.

In one embodiment, the sensing unit may include a frame-type or protrusion-type dielectric portion, while the reference unit may include a bulk-form dielectric or no dielectric portion at all.

The pressure change ΔP may be calculated according to Equation 1 (to be defined in the specification).

$$Δ {C}_{M} = ( {ε}_{1} \frac{A}{Δ {d}_{1}} - {Δε}_{1} \frac{A}{d} ) - ( {ε}_{2} \frac{A}{Δ {d}_{2}} - {Δε}_{2} \frac{A}{d} ) \qquad [ Equation 1 ]$$

In Equation 1:
ε₁ denotes the dielectric constant of the sensing unit,
A represents the area of the first electrode of the sensing unit and the second electrode of the reference unit,
Δd₁ is the variation in the distance between the first and second electrodes of the sensing unit,
Δε₁ denotes the variation in the dielectric constant of the sensing unit due to noise,
d is the distance between the first and second electrodes of the sensing unit and between the third and second electrodes of the reference unit,
ε2 denotes the dielectric constant of the reference unit,
Δd₂ is the variation in the distance between the third and second electrodes of the reference unit, and
Δε₂ represents the variation in the dielectric constant of the reference unit due to noise

In one embodiment, the sensing unit and the reference unit may be repeatedly stacked one or more times to form a multilayer structure.

According to another aspect of the present invention, the biodegradable sensor for pressure measurement as described in the present embodiment may be applied to medical implants.

### Advantageous Effects

As described above, the present invention provides the following advantages: According to the present invention, a biodegradable sensor can be provided, which is implantable in vivo for continuous pressure monitoring and is biodegraded after a predetermined period, thereby eliminating the need for surgical removal.

In addition, the present invention provides a manufacturing method for producing such a biodegradable pressure sensor.

Furthermore, by including multiple biodegradable sensors, the invention enables the construction of a composite sensor assembly capable of reducing signal noise and enhancing measurement reliability.

### BRIEF DESCRIPTION OF THE DRAWINGS

FIG. 1 is a schematic view illustrating a biodegradable sensor and its implantation in the brain according to an embodiment of the present invention.

FIG. 2 illustrates a manufacturing process of the biodegradable sensor according to an embodiment of the present invention.

FIG. 3 is a cross-sectional view of a monolayer biodegradable sensor fabricated with a polymer dielectric of a specific shape, according to an embodiment of the present invention.

FIG. 4 is a cross-sectional view of a multilayer biodegradable sensor fabricated with a polymer dielectric of a different shape, according to an embodiment of the present invention.

FIG. 5 is an image showing polymer dielectrics with horn-shaped structures arranged at varying intervals, according to an embodiment of the present invention.

FIGS. 6 and 7 are schematic views illustrating a composite sensor assembly according to an embodiment of the present invention.

FIG. 8 shows results of noise and sensitivity measurements of the monolayer biodegradable sensor in an ambient environment according to an embodiment of the present invention.

FIG. 9 shows results of noise and sensitivity measurements of the monolayer biodegradable sensor in a PBS (phosphate-buffered saline) environment according to an embodiment of the present invention.

FIG. 10 shows results of noise and sensitivity measurements of the multilayer biodegradable sensor in an ambient environment according to an embodiment of the present invention.

FIG. 11 shows results of noise and sensitivity measurements of the multilayer biodegradable sensor in a PBS environment according to an embodiment of the present invention.

FIG. 12 shows results of noise and pressure variation measurements in an animal brain using the composite sensor assembly according to an embodiment of the present invention.

FIG. 13 shows the noise reduction effect of the composite sensor assembly according to an embodiment of the present invention.

### MODES OF THE INVENTION

An exemplary embodiment of the present invention will be described with reference to the accompanying drawings, and an object and the configuration, and the features of the present invention will be understood well through the detailed description.

The exemplary embodiment described above is only to describe exemplary embodiment of the present invention and is not limited to the exemplary embodiment, and various modifications and variations are possible by those skilled in the art within the spirit and claims of the present invention, and it will be said that the modifications and variations fall within the scope of the technical rights of the present invention.

The advantages and features of the present invention, and the manner of achieving them, will be more clearly understood by reference to the following detailed description of exemplary embodiments in conjunction with the accompanying drawings. However, the invention is not limited to the embodiments set forth below and may be embodied in various other forms. Unless otherwise specified, all numerical expressions, values, and/or parameters used to describe quantities, conditions, and compositions in the present invention should be understood as approximations that reflect the inherent variability in measurements, and should therefore be interpreted as being preceded by the term “about” in all cases.

Moreover, when numerical ranges are disclosed herein, such ranges are intended to be continuous and to include every value between the minimum and maximum values set forth, unless expressly stated otherwise. Further, unless otherwise specified, all values within a stated range are to be considered as specifically disclosed.

When a range refers to integers, it should be understood to include every individual integer within the stated range unless otherwise indicated. For example, a range of “5 to 10” should be understood to include 5, 6, 7, 8, 9, and 10, as well as any subranges such as 6 to 10, 7 to 9, etc. This also includes decimal values and subranges, such as 5.5 to 8.5 or 6.5 to 9.

Likewise, a range such as “10% to 30%” includes all percentage values between 10% and 30%, including 11%, 12%, 13%, up to 30%, and also includes subranges such as 10% to 15%, 12% to 18%, and 20% to 30%, as well as intermediate values such as 10.5%, 15.5%, and 25.5%.

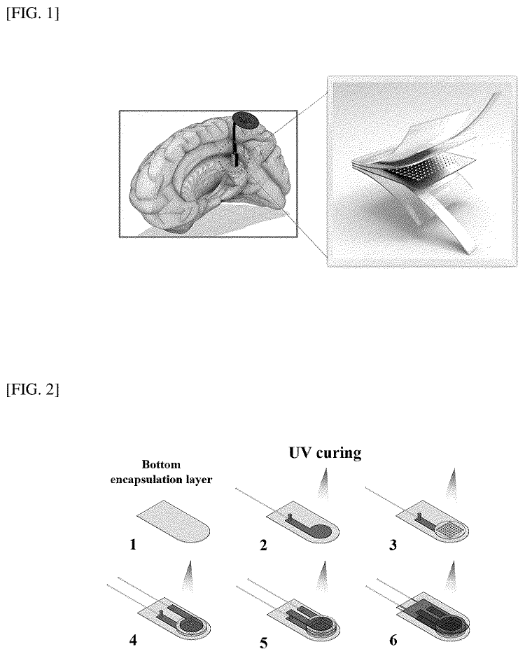

FIG. 1 is a schematic view illustrating a composite sensor assembly including a biodegradable sensor for pressure measurement according to an embodiment of the present invention. The electrodes, dielectric portion, and packaging of the sensor are all composed of materials that are biodegradable in vivo. Therefore, after implantation into the body and subsequent pressure monitoring, the sensor is naturally decomposed over time, eliminating the need for a separate surgical removal procedure.

According to one aspect of the present invention, a biodegradable sensor for pressure measurement may include: a first electrode comprising a first metal that is biodegradable in vivo; a second electrode comprising a second metal that is biodegradable in vivo; and a dielectric portion disposed between the first and second electrodes and comprising a biodegradable polymer dielectric, wherein the pressure is measured based on a change in the distance between the first and second electrodes.

In one embodiment, the biodegradable sensor may operate based on a capacitive sensing mechanism, also referred to as a capacitive pressure sensor. The sensor may have a structure analogous to a parallel plate capacitor, in which a dielectric is interposed between two facing electrodes. The capacitance of the sensor changes in response to variations in the inter-electrode distance caused by external pressure. This change in capacitance can be translated into an electrical signal representing the pressure. This capacitive sensing approach enables the sensor to be fabricated in various forms through a relatively simple process, allowing customization of the sensor's geometry and sensitivity according to the application site or pressure range.

The dielectric portion may take various forms between the first and second electrodes.

The dielectric layer may be deformed depending on the degree of compression by the electrodes, enabling the sensor to detect pressure at the implantation site with high sensitivity. The dielectric structure is not limited to a specific form and may include: a bulk form (completely filled), a frame form having an internal cavity, or a protruded form (e.g., pillar or horn-shaped structures).

### Bulk Form:

In one embodiment, the bulk-type dielectric may be provided in a thin-film form, which is compressed to measure pressure. The thickness of the bulk form may range from: 50 nm to 200 μm, or 60 nm to 150 nm, or 200 nm to 400 nm, or 30 μm to 200 μm, or 40 μm to 60 μm, or 100 μm to 120 μm. Preferably, if the dielectric is composed of organic materials, the bulk thickness may be in the range of 10 μm to 200 μm, whereas for inorganic materials, it may range from 50 nm to 500 nm.

### Frame Form:

In another embodiment, the frame-type dielectric may include a cavity surrounded by a dielectric frame. The frame may comprise: a first surface facing the first electrode; a second surface facing the second electrode, spaced from the first surface across the cavity; and a third surface connecting the edges of the first and second surfaces.

The cavity may contain, for example, air, and the pressure is measured by the degree of deformation of the first and second surfaces pressing on the cavity.

While reducing the thickness of the dielectric frame increases sensitivity, excessive thinning may reduce durability. The dielectric frame may have a thickness of: 10 μm to 150 μm, or 30 μm to 50 μm, or 80 μm to 100 μm.

The spacing between the first and second surfaces affects both sensitivity and measurable pressure range. A larger gap increases the range but decreases sensitivity and increases device size. The gap may be: 50 μm to 200 μm, or 130 μm to 150 μm, or 170 μm to 200 μm.

### Protruded Form:

The protruded-type dielectric may comprise raised structures with circular or polygonal bottom surfaces arranged at regular intervals. The protrusions may resemble horns or pillars and collectively respond to external pressure. The interval between the protrusion bases may be: 10 μm to 500 μm, or 30 μm to 50 μm, or 80 μm to 100 μm, or 180 μm to 200 μm, or 280 μm to 300 μm, or 360 μm to 400 μm.

If spacing is too narrow, sensor sensitivity may decrease; if too wide, structural durability may be compromised.

The height of the protrusions may be: 20 μm to 70 μm, or 30 μm to 35 μm, or 60 μm to 70 μm.

The overall thickness of the dielectric portion including the protrusions may be: 100 μm to 500 μm, or 200 μm to 300 μm, or 210 μm to 230 μm.

An excessively thick dielectric may allow a wider measurable pressure range, but may reduce sensitivity and complicate surgical implantation.

### Dielectric Constant:

The dielectric constant of the dielectric portion may be: 0.1 pF/m to 0.5 pF/m, or preferably 0.2 pF/m to 0.3 pF/m.

A dielectric constant exceeding 0.5 pF/m may enhance sensitivity but limit the selection of materials with adequate biodegradability and biocompatibility. If the dielectric constant is greater than that of the surrounding environment, the signal-to-noise ratio (SNR) may improve. Conversely, if it is lower than 0.1 pF/m, sensitivity may significantly decrease.

### Electrode Materials:

The first electrode may comprise at least one metal selected from the group consisting of magnesium (Mg), zinc (Zn), tungsten (W), and molybdenum (Mo). The second electrode may also comprise at least one of the same metals. In some embodiments, the first and second electrodes may be formed from the same metal.

In one embodiment, the dielectric portion may comprise a polymeric dielectric, which may include at least one polymer selected from the group consisting of: poly(lactic-co-glycolic acid) (PLGA), poly(butanedithiol pentenoic anhydride) (PBTPA), polyglycolic acid (PGA), polycaprolactone (PCL), poly(glycerol sebacate) (PGS), polylactic acid (PLA), gelatin methacryloyl (GelMA), gelatin methacryloyl-polyethylene glycol diacrylate (GelMA-PEGDA), and polyurethane (PU).

In one embodiment, when the polymeric dielectric comprises PBTPA (poly(butanedithiol pentenoic anhydride)), the performance characteristics of the sensor may be tuned by adjusting the synthesis ratio of its precursors. The PBTPA may be synthesized from the following monomers: 4-pentenoic anhydride (4PA), 1,3,5-triallyl-1,3,5-triazine-2,4,6(1H,3H,5H)-trione (TTT), and 1,4-butanedithiol (BDT).

When the relative proportion of TTT and BDT is low, the resulting polymer may exhibit reduced mechanical strength but improved sensitivity. Conversely, when the proportion of TTT and BDT is high, the resulting polymer may possess a hydrophobic surface and exhibit slower biodegradation.

The molar ratio among the precursors 4PA, TTT, and BDT may be adjusted depending on the application or functional requirements of the biodegradable sensor. For example, the precursors may be included in a molar ratio ranging from 1:1:2.5 to 1:4:7.

In one embodiment, the biodegradable sensor may be configured to degrade in vivo over a period ranging from 30 hours to 60 days. The degradation period may be adjusted by selecting the materials of the biodegradable sensor based on the clinical duration required for pressure monitoring within the body.

The biodegradable sensor may further comprise a multilayer capacitor formed by laminating multiple sets of electrodes and dielectric portions.

According to another aspect of the present invention, a method of manufacturing at least one biodegradable sensor having the aforementioned characteristics may include the steps of: forming a first electrode by applying a paste containing a first metal or by attaching a film comprising the first metal; forming a dielectric portion including a polymer dielectric over the first electrode; and forming a second electrode over the dielectric portion by applying a paste containing a second metal or attaching a film comprising the second metal.

When the first or second electrode is formed by attaching a film, the film may be, for example, a metal film having a thickness of 3 μm to 20 μm, though it is not limited thereto.

The manufacturing method may further include a step of encapsulating the biodegradable sensor using a material that is biodegradable in vivo.

Each step in the manufacturing process may include curing the applied materials within a moisture-limited environment. For example, the curing may be performed by UV irradiation, which can significantly shorten the processing time compared to conventional methods and improve production efficiency. Each curing step may take 1 to 8 minutes, and preferably 5 to 7 minutes.

In one example, the step of forming the dielectric portion may involve creating a bulk form (completely filled structure). The dielectric portion in this case may be formed by one or more of the following methods: chemical vapor deposition (CVD), sputtering, screen printing, comma printing, or photolithography.

Alternatively, the dielectric portion may be formed in a frame form having an internal cavity. This may include: forming a first surface facing the first electrode by printing or applying a polymer dielectric; forming a third surface along the edge of the first surface, thereby defining a cavity enclosed by the third surface; and forming a second surface over the third surface by laminating or printing a polymer dielectric to face the second electrode.

In one embodiment, the frame-type dielectric portion with an internal cavity may be formed by screen printing. Specifically, the process may include: printing the polymer dielectric via screen printing to form the first surface and then curing it; printing the dielectric material on the edges of the first surface to form the third surface, followed by curing; and finally, placing a polymer dielectric film over the third surface and curing it to form the second surface.

In another example, the dielectric portion may be formed in a protruded structure (e.g., micro-pillar or cone-like geometry). The protruded dielectric may be fabricated by pouring a polymer dielectric into a mold having the desired protrusion shape, followed by curing. After curing, the mold is removed, and the resulting dielectric structure is placed on top of the first electrode, thereby forming the dielectric portion. A second electrode is then formed over the dielectric portion.

In one embodiment, the protruded dielectric structure may be fabricated using photolithography. For instance, the process may comprise the following steps: Oxidizing a silicon (Si) substrate to form a SiO₂ thin film; Patterning the SiO₂ film using a buffer oxide etchant (BOE) to produce a mask; Etching the underlying silicon using potassium hydroxide (KOH) through the SiO₂ mask to create protruded features; Cleaning and applying a silane-based self-assembled monolayer (SAM) to the etched surface to form a mold with defined protrusion shapes; Screen printing the polymer dielectric into the mold and curing it to produce the protruded dielectric structure; Separating the cured dielectric from the mold and transferring it onto the first electrode to form the dielectric portion. After the dielectric portion is positioned, a second electrode is formed over the protruded structure to complete the capacitive sensor configuration.

The first electrode may comprise a metal, such as magnesium (Mg), zinc (Zn), tungsten (W), or molybdenum (Mo). Similarly, the second electrode may comprise at least one of the same metals. Preferably, the first and second electrodes are composed of the same metal to ensure uniform degradation and electrical compatibility.

In one embodiment, the polymer dielectric may comprise at least one polymer selected from the group consisting of: poly(lactic-co-glycolic acid) (PLGA), poly(butanedithiol pentenoic anhydride) (PBTPA), polyglycolic acid (PGA), polycaprolactone (PCL), poly(glycerol sebacate) (PGS), polylactic acid (PLA), gelatin methacryloyl (GelMA), gelatin methacryloyl-polyethylene glycol diacrylate (GelMA-PEGDA), and polyurethane (PU).

In a preferred embodiment, when the polymer dielectric is PBTPA (poly(butanedithiol pentenoic anhydride)), the properties of the sensor may be tuned by adjusting the molar ratios of its precursors. The PBTPA may be synthesized using: 4-pentenoic anhydride (4PA), 1,3,5-triallyl-1,3,5-triazine-2,4,6(1H,3H,5H)-trione (TTT), and 1,4-butanedithiol (BDT) as monomeric precursors.

When the ratio of TTT and BDT is low, the resulting polymer tends to exhibit lower mechanical strength but enhanced sensitivity due to increased flexibility. Conversely, when the ratio of TTT and BDT is high, the polymer surface may become more hydrophobic, which can slow the degradation rate in vivo.

The specific molar ratio of 4PA:TTT:BDT may be selected depending on the intended use and target lifetime of the biodegradable sensor. For example, the precursors may be used in a molar ratio ranging from 1:1:2.5 to 1:4:7.

In one embodiment, the biodegradable sensor may be configured to form a multilayer capacitor by repeating the steps of forming the dielectric portion and the second electrode after forming the second electrode. The resulting multilayer structure may provide higher sensitivity compared to a single-layer configuration.

The present invention may also include a composite sensor assembly comprising at least one biodegradable sensor having one or more of the aforementioned features.

Specifically, the composite sensor assembly may comprise: a sensing unit including a biodegradable sensor configured to produce a variable electrical signal in response to changes in external pressure; and a reference unit including a third electrode and configured to produce a constant electrical signal, irrespective of pressure changes.

The composite sensor assembly may detect external pressure changes by comparing the electrical signal measured by the sensing unit with that of the reference unit.

In one embodiment, the sensing unit and the reference unit may be identical in structure (i.e., twin-type layout), or alternatively, they may have different sensor configurations.

The sensing unit is designed to detect external pressure by measuring an electrical signal that varies with the compression or deformation of the dielectric portion. For example, the dielectric portion of the sensing unit may be configured such that its thickness or width changes in response to external pressure, leading to a variation in the distance between the first and second electrodes. The dielectric portion may be formed in a frame structure with an internal cavity or a protruded structure, although other forms are also possible.

The reference unit functions as a reference sensor, providing a constant electrical signal under unchanged pressure conditions. The dielectric portion of the reference unit may be in the form of a bulk dielectric, or in some cases, it may consist solely of electrodes, such as a configuration where only the second and third electrodes are present without any dielectric layer. The reference unit produces a stable signal that serves as a baseline against which the variable signal from the sensing unit can be compared.

By incorporating the reference unit, the composite sensor assembly can effectively eliminate noise and achieve a higher signal-to-noise ratio (SNR) than a single sensing unit. The system calculates the difference between the signals from the sensing unit and the reference unit to determine pressure changes while compensating for noise.

The detection of external pressure changes may be expressed by Equation 1 (defined below).

$$Δ {C}_{M} = ( {ε}_{1} \frac{A}{Δ {d}_{1}} - {Δε}_{1} \frac{A}{d} ) - ( {ε}_{2} \frac{A}{Δ {d}_{2}} - {Δε}_{2} \frac{A}{d} ) \qquad [ Equation 1 ]$$

In Equation 1, ΔCM represents the net change in capacitance resulting solely from pressure variation, i.e., the electrical signal corresponding to the actual pressure change after noise elimination.

The first term on the right-hand side, ΔCactive, denotes the capacitance change measured by the sensing unit, which reflects both pressure-induced variation and potential environmental or system noise.

The second term on the right-hand side, ΔCnonactive, represents the capacitance change measured by the reference unit, which is not influenced by external pressure but may reflect environmental drift or background noise.

By subtracting ΔCnonactive from ΔCactive, ACM isolates the signal component purely attributable to external pressure, thereby enabling more accurate and noise-compensated pressure detection.

In Equation 1, the variables are defined as follows:
ε₁: Dielectric constant of the sensing unit
A: Area of the first electrode of the sensing unit and the second electrode of the reference unit
Δd₁: Change in the distance between the first and second electrodes of the sensing unit due to external pressure
Δε₁: Change in dielectric constant in the sensing unit due to noise
d: Baseline distance between the first and second electrodes of the sensing unit, and between the third and second electrodes of the reference unit
ε₂: Dielectric constant of the reference unit
Δd₂: Change in the distance between the third and second electrodes of the reference unit due to environmental effects
Δε₂: Change in dielectric constant in the reference unit due to noise

Each component of Equation 1 may be further expressed by Equations 2 and 3, and accordingly, ΔCM can ultimately be expressed by Equation 4.

The reference unit may be a region in which no variation in electrical signal occurs in response to pressure change. Accordingly, since the distance between the third electrode and the second electrode of the reference unit does not change under pressure, this relationship can be expressed as Equation 2.

Among the elements of Equation 1, Equation 5 represents the noise component measured in the sensing unit, while Equation 6 represents the noise component measured in the reference unit.

The noise measured in the sensing unit and the reference unit is assumed to be nearly identical, and thus can be expressed using Equation 5.

$${ε}_{2} \frac{A}{Δ {d}_{2}} \approx 0 \qquad [ Equation 2 ]$$

$${Δε}_{1} \frac{A}{d} \approx {Δε}_{2} \frac{A}{d} \qquad [ Equation 3 ]$$

$${ε}_{1} \frac{A}{Δ {d}_{1}} \qquad [ Equation 4 ]$$

$${Δε}_{1} \frac{A}{d} \qquad [ Equation 5 ]$$

$${Δε}_{2} \frac{A}{d} \qquad [ Equation 6 ]$$

The sensing unit and the reference unit may be arranged side by side or alternatively stacked to form the composite sensor assembly. When the sensing and reference units are stacked, they may be laminated repeatedly one or more times.

By incorporating a reference sensor in addition to the primary pressure sensing unit, the composite sensor assembly of the present invention can fundamentally eliminate electrostatic measurement errors commonly associated with conventional capacitive pressure sensors.

Accordingly, the biodegradable sensor of the present invention, which exhibits excellent biocompatibility and environmental sustainability, can be applied not only to implantable medical devices but also to a wide range of applications that require in vivo pressure monitoring with biodegradability.

The following examples and comparative examples are provided to further illustrate the preferred embodiments of the present invention. However, these examples are not intended to limit the scope of the present invention in any way.

### Manufacturing Example 1

FIG. 2 is a schematic illustration showing the manufacturing process of a biodegradable sensor according to one embodiment of the present invention.

A first electrode was formed on the bottom layer of a biodegradable capsule, and was cured using UV irradiation. The first electrode was fabricated by cutting a molybdenum (Mo) film with a thickness of 10 μm.

The bottom layer of the capsule was composed of a biodegradable photopolymer, specifically PBTPA (poly(butanedithiol pentenoic anhydride)), prepared by mixing 4-pentenoic anhydride (4PA), triallyl triazine trione (TTT), and 1,4-butanedithiol (BDT) in a molar ratio of 1:4:7.

Next, a dielectric layer was formed over the first electrode using a biodegradable polymer dielectric and subsequently UV-cured. The dielectric layer was composed of PBTPA prepared by mixing 4PA, TTT, and BDT in a molar ratio of 1:1:2.5.

A second electrode was then formed over the dielectric portion. The structure was encapsulated with a top layer made from the same biodegradable PBTPA material as the bottom layer. Each layer formation step involved UV curing.

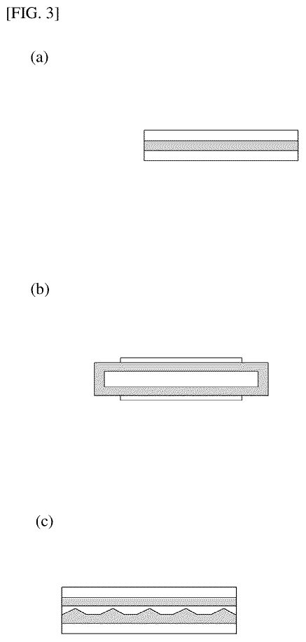

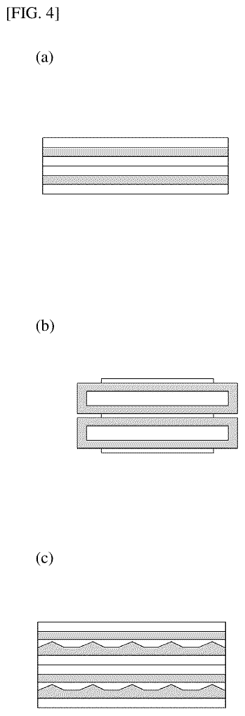

The dielectric portion was fabricated in three different configurations, both single-layer and multilayer, as schematically illustrated in FIGS. 3 and 4, respectively.

In the single-layer configuration, the total sensor height was approximately 400 μm. In the multilayer configuration, the total sensor height was approximately 550 μm.

### Manufacturing Example 2

A composite sensor assembly was fabricated by incorporating the biodegradable sensor described above. The assembly included both a sensing unit and a reference unit.

### Example 1

In Manufacturing Example 1, the step of forming the dielectric portion was modified.

Specifically, the polymer dielectric was formed into a thin-film structure via screen printing. All other processes remained the same as described in Manufacturing Example 1.

### Example 2

In this example, the dielectric portion was fabricated using a modified procedure, while all other steps remained identical to Manufacturing Example 1.

A screen-printing mask was placed on the first electrode to form a first surface, and a biodegradable polymer was screen-printed onto the mask and UV-cured for 5 minutes.

To form the cavity structure, an additional screen-printing mask was positioned around the edge of the first surface, and the biodegradable polymer was again screen-printed and UV-cured for 5 minutes to form a third surface.

After removing the cavity-forming mask, a biodegradable polymer film was placed over the third surface and cured to form a second surface, thereby completing the dielectric structure with a frame-type cavity.

### Example 3

In this example, the dielectric portion was fabricated in a pyramidal protruded form, while all other procedures followed Manufacturing Example 1.

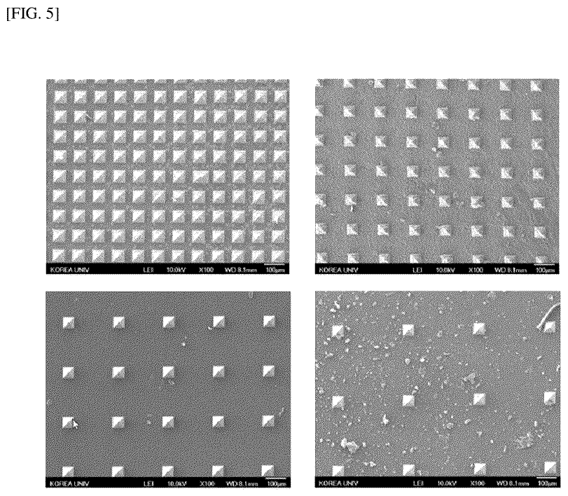

As shown in FIG. 5, the dielectric layer was fabricated with varying inter-protrusion spacing, and the spacing values for each example are listed in Table 1.

To fabricate the protruded dielectric: A silicon (Si) wafer was thermally oxidized in a furnace to form a SiO₂ thin film. A photolithography process was applied using buffer oxide etchant (BOE) to pattern the SiO₂ film and form a mask. The patterned SiO₂ mask was etched in potassium hydroxide (KOH) to produce inverted pyramidal features, followed by DI water cleaning. The etched silicon substrate was treated with silane for self-assembled monolayer (SAM) formation, resulting in an inverted pyramidal silicon mold. A polymer dielectric was screen-printed into the mold and UV-cured, then released from the mold and transferred onto the electrode to complete the protruded dielectric portion.

TABLE 1

| Sample ID | Base-to-base spacing of protruded dielectric (μm) |
|---|---|
| Example 3-1 | 40 |
| Example 3-2 | 100 |
| Example 3-3 | 200 |
| Example 3-4 | 300 |

### Example 4

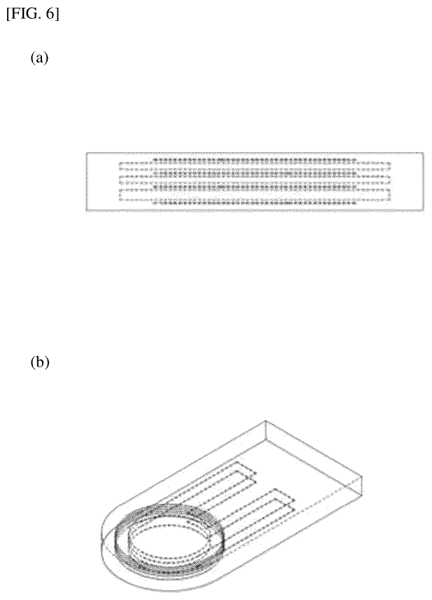

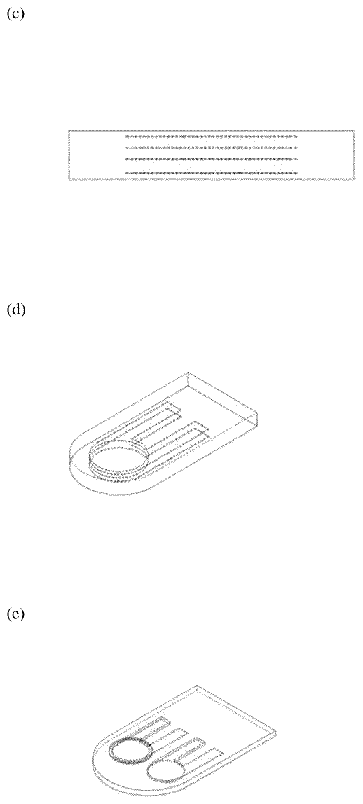

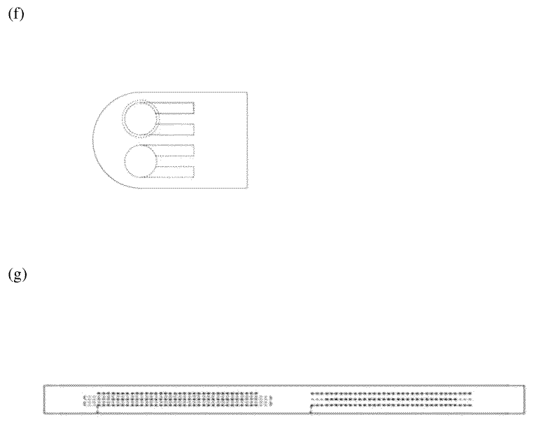

A composite sensor assembly was fabricated using a multilayer structure of the biodegradable sensor produced in Example 2 as the sensing unit. The resulting configuration is schematically shown in FIGS. 6(a) and 6(b).

Similarly, a reference unit was fabricated by laminating multiple layers of the biodegradable sensor produced in Example 1, as illustrated in FIGS. 6(c) and 6(d). Both the sensing and reference units were fabricated with identical specifications, ensuring consistency in structure and size. The two units were placed side by side, and pressure was applied independently to each. By calculating the difference between the measured signals, the system was able to accurately determine the true pressure value, while compensating for environmental noise and measurement drift.

### Example 5

FIGS. 6(e), 6(f), and 6(g) illustrate a composite sensor assembly in which a reference unit was formed using the biodegradable sensor of Example 1, and a sensing unit was formed using the biodegradable sensor of Example 2.

The two units were arranged side by side and packaged together to form the final composite sensor assembly. This configuration enables differential measurement of pressure using paired sensing and reference sensors with distinct dielectric structures.

### Example 6

A composite sensor assembly was fabricated by combining: a sensing unit comprising the biodegradable sensor of Example 2, and a reference unit that does not include a dielectric portion, but comprises electrodes only.

The sensing and reference units were laminated and encapsulated into a single device.

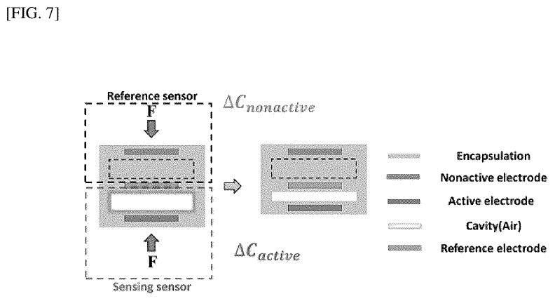

This configuration is schematically illustrated in FIG. 7.

In this embodiment: The reference unit measures the electrical signal between the second electrode (labeled as “Reference Electrode” in FIG. 7) and the third electrode (labeled as “Nonactive Electrode” in FIG. 7). The encapsulation material surrounding the composite sensor assembly serves as a dielectric medium between the second and third electrodes.

The region labeled as “Nonactive Space” in FIG. 7 indicates that no dedicated dielectric layer (such as the polymer dielectric used in previous examples) is present between the reference electrodes. The second and third electrodes are spaced apart at a fixed distance, enabling the reference unit to output a stable electrical signal that is not influenced by external pressure.

### Experimental Example

The biodegradable sensor fabricated in Example 4-4 was tested using a pressure application system based on pneumatic deformation to evaluate its output response to varying pressure conditions. The purpose of the test was to compare the sensor's performance against that of a commercial pressure sensor.

The time-sensitive pressure sensor was implemented using the sensor fabricated in the example. The sensor output was measured using an EVAL-AD7746 Evaluation Board (Analog Devices™)

As a reference, a commercial pressure sensor, specifically the ABP DRR model (Honeywell), was used for comparison under the same pressurization and depressurization conditions.

The results confirmed the capability of the biodegradable pressure sensor to detect pressure changes with an output pattern comparable to that of the commercial sensor, validating its potential use in real-time, biodegradable sensing applications.

### 1. Characteristics of Single-Layer Capacitive Biodegradable Pressure Sensor

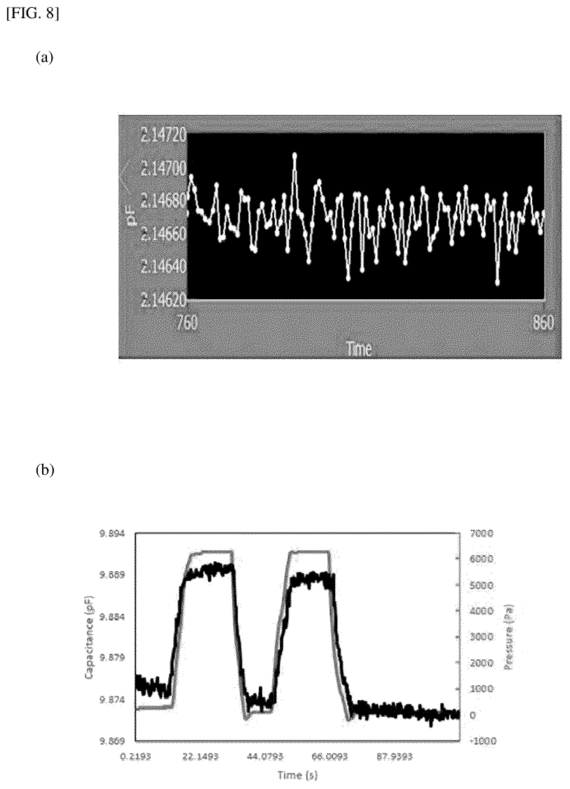

FIG. 8(a) shows the noise level of the single-layer capacitive pressure sensor when kept stationary in ambient air without applied pressure. The measured noise was approximately 0.7 femtofarads (fF), indicating excellent electrical stability under static air conditions.

FIG. 8(b) presents the pressure response of the biodegradable pressure sensor (black curve) and a commercial reference pressure sensor (red curve) during periodic pressurization and depressurization cycles using air. The biodegradable sensor output was measured using the EVAL-AD7746 Evaluation Board (Analog Devices™), and the reference sensor used was the ABP DRR model (Honeywell™)

Both sensors exhibited similar response trends under cyclic pressure changes. The calculated sensitivity of the biodegradable sensor was 2.17×10⁻⁶ pF/Pa.

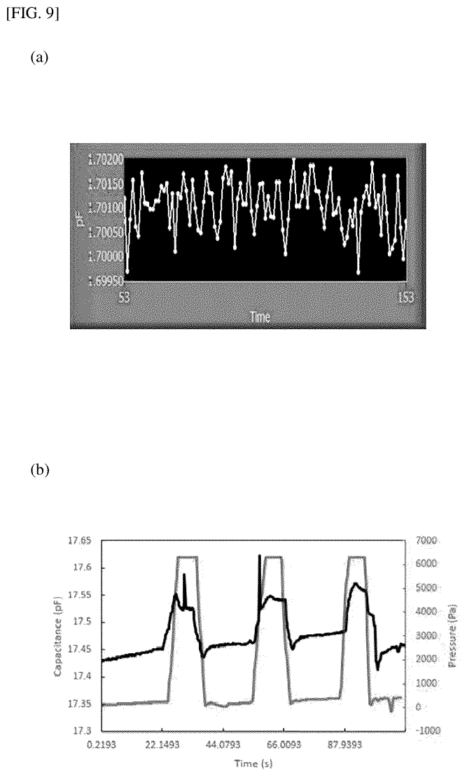

FIG. 9(a) illustrates the noise behavior of the same biodegradable pressure sensor when immersed in phosphate-buffered saline (PBS) without pressure loading. The noise level was observed to be approximately 1.5 fF, which is slightly higher than in air due to dielectric perturbations in aqueous conditions.

FIG. 9(b) shows the output response curves of the biodegradable sensor (black line) and the commercial reference sensor (red line) under pressure cycling in PBS. Both sensors demonstrated similar capacitive response trends. The sensitivity of the biodegradable sensor in PBS was calculated to be 4.00×10⁻⁶ pF/Pa.

These results confirm that the single-layer biodegradable pressure sensor provides stable and reliable capacitive measurements, showing sensitivity values comparable to those of commercial sensors, and maintains performance in both air and physiological (PBS) environments.

### 2. Characteristics of Multilayer Capacitive Biodegradable Pressure Sensor

In order to evaluate the performance of the biodegradable pressure sensor fabricated in a multilayer structure, sensitivity and noise characteristics were analyzed under two environmental conditions: ambient air and phosphate-buffered saline (PBS).

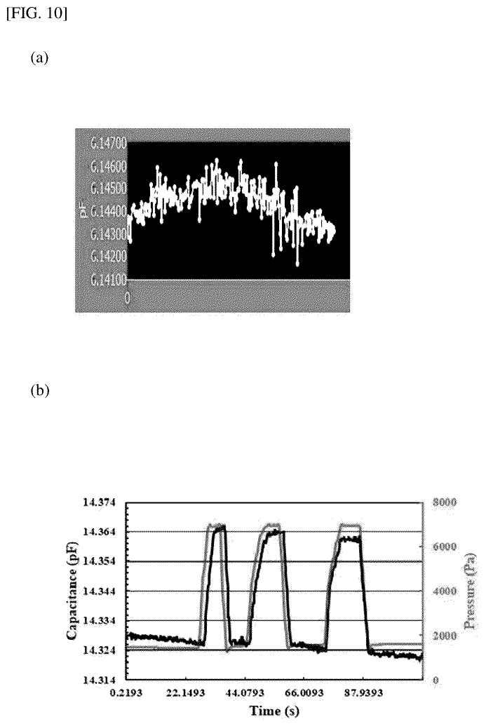

FIG. 10(a) shows the noise profile of a multilayer capacitive biodegradable pressure sensor when left stationary in ambient air. The measured noise level was approximately 4 femtofarads (fF), which indicates a slightly higher baseline fluctuation compared to the single-layer sensor. This increase in noise may be attributed to the additional interfaces between stacked layers.

FIG. 10(b) illustrates the output responses of the transient (biodegradable) multilayer pressure sensor and a commercial pressure sensor (ABP DRR by Honeywell™) under repeated pressurization and depressurization in air. In the figure, the black line represents the biodegradable sensor, and the red line represents the commercial sensor.

The measured sensitivity of the biodegradable multilayer sensor was 6.55×10⁻⁶ pF/Pa, which shows a notable improvement over the single-layer sensor (2.17×10⁻⁶ pF/Pa). The sensor exhibited a response pattern comparable to that of the commercial device, confirming its reliability under dynamic pressure environments.

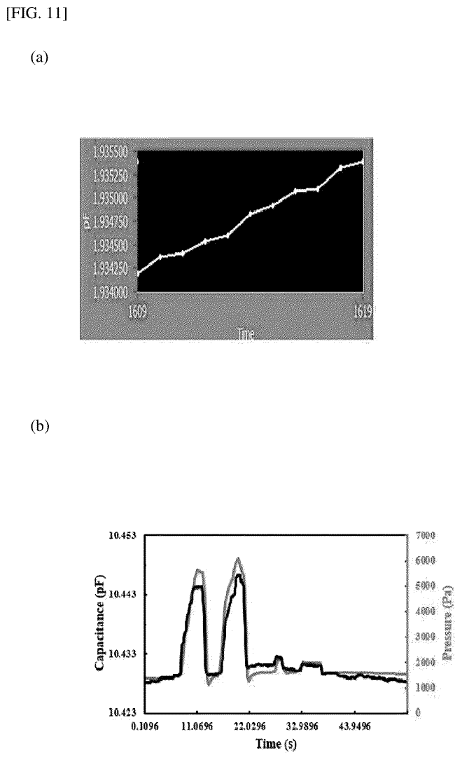

FIG. 11(a) displays the noise level of the same multilayer sensor when submerged and left undisturbed in PBS solution. The measured noise was 0.0001 pF (100 fF), indicating a relatively stable baseline even in a liquid medium. This demonstrates the feasibility of deploying the sensor in bio-relevant aqueous environments.

FIG. 11(b) presents the output changes of the biodegradable and commercial sensors under pressure fluctuations in PBS. Again, the black line denotes the biodegradable sensor, while the red line indicates the commercial sensor. The response patterns closely match across both devices.

The calculated sensitivity of the biodegradable multilayer sensor in PBS was 7.10×10⁻⁶ pF/Pa, further confirming improved sensitivity in physiological conditions.

The sensitivity values in FIGS. 8 through 11 were all measured within two internal pressure ranges.

### [Experimental Example 2: In Vivo Performance of the Biodegradable Composite Pressure Sensor]

In order to evaluate the in vivo functionality and signal reliability of the composite biodegradable pressure sensor, the sensor assembly fabricated in Preparation Example 2-2 was implanted into a live animal model.

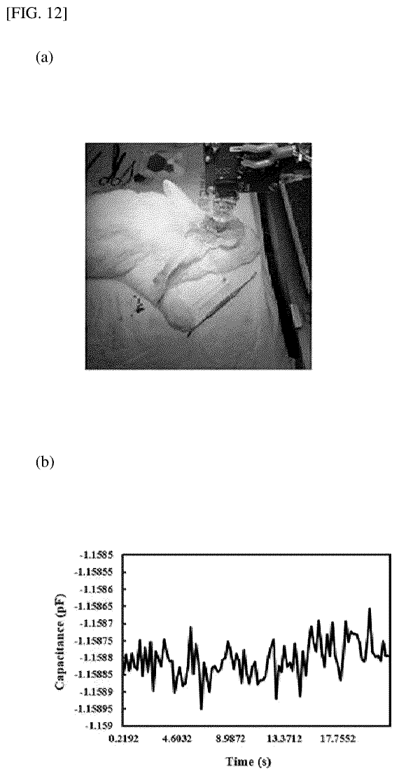

FIG. 12(a) shows an image of the sensor assembly surgically implanted inside the cranium of a test animal. After implantation, controlled mechanical pressure was repeatedly applied to the abdominal region to induce corresponding intracranial pressure changes.

FIG. 12(b) presents the noise data measured when the biodegradable composite pressure sensor was placed inside the animal body. The observed noise level was 0.2 femtofarads (fF), indicating excellent signal stability under biological conditions.

FIG. 12(c) compares the output signals of the biodegradable sensor and a commercial pressure sensor in response to physical force applied to the test animal. The biodegradable sensor demonstrated a sensitivity of 4.5×10⁻⁶ pF/Pa, closely tracking the pressure variations detected by the commercial sensor.

Furthermore, the Signal-to-Noise Ratio (SNR) under in vivo conditions at a reference pressure of 1000 Pa was calculated to be 30, representing a significant enhancement compared to an SNR of 0.38 obtained when pressure was measured using the biodegradable sensor alone without a reference channel. This reflects a ~79-fold improvement in signal clarity and reliability by employing a differential measurement strategy with a reference sensor.

The output trends between the biodegradable sensor and the commercial pressure sensor under physiologically induced pressure variations were found to be highly consistent, confirming the utility and accuracy of the developed sensor for biomedical applications.

[Experimental Example 3: Evaluation of Capacitance Variation with and without Reference Sensor]

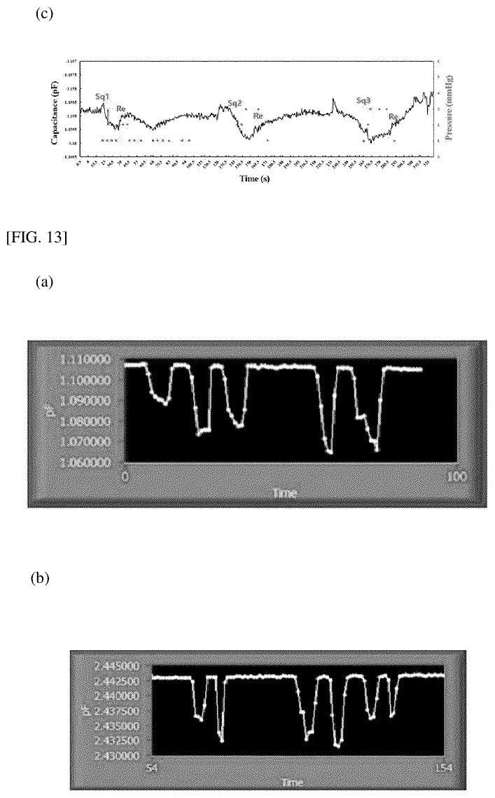

To assess the effect of employing a reference sensor on signal clarity, a composite sensor assembly fabricated according to Preparation Example 2-3 was evaluated for its capacitance variation response under external stimulus. The measurement results are illustrated in FIG. 13.

As shown in FIG. 13(a), when a hand was brought near the sensor without any applied mechanical pressure and without using a reference sensor, a capacitance variation of approximately 40 femtofarads (fF) was observed. This variation was attributed primarily to environmental interference such as electromagnetic coupling or stray capacitance from the human body.

In contrast, FIG. 13(b) demonstrates the response when the same stimulus was applied under identical conditions to a sensor assembly integrated with a reference sensor. In this case, the capacitance variation was significantly reduced to approximately 11 fF.

This result indicates that inclusion of a reference sensor effectively cancels out or compensates for non-pressure-related environmental disturbances. Specifically, the implementation of the reference sensor led to a ~¼ reduction in noise amplitude, thereby enhancing the signal-to-noise ratio (SNR) and measurement accuracy of the system.

This experiment validates that the differential signal acquisition approach using a reference sensor enables improved stability and precision by suppressing external noise components. As a result, the system demonstrates higher reliability for detecting pressure-dependent capacitance variations in both ambient and dynamic settings.

It will be understood by those skilled in the art that the present invention can be implemented in other specific forms without departing from the spirit or essential characteristics of the invention. Therefore, the embodiments disclosed above are to be considered in all respects as illustrative and not restrictive. The scope of the invention is indicated by the claims rather than by the foregoing description, and all changes or modifications derived from the meaning, scope, and equivalents of the claims are to be construed as being included within the scope of the invention.

### INDUSTRIAL APPLICABILITY

The biodegradable sensor according to the present invention has excellent biocompatibility and environmental compatibility and can be biodegraded in vivo, so it can be applied to medical implants implanted in the body, etc., and can be expanded to a variety of application fields.

1. A biodegradable sensor for pressure measurement, comprising: a first electrode comprising a first metal degradable in vivo; a second electrode comprising a second metal degradable in vivo; and a dielectric portion disposed between the first electrode and the second electrode, the dielectric portion comprising a polymer degradable in vivo, wherein the sensor measures pressure based on a variation in spacing between the first electrode and the second electrode.

2. The biodegradable sensor of claim 1, wherein the sensor measures pressure using a capacitive sensing method.

3. The biodegradable sensor of claim 1, wherein the dielectric portion is in a form selected from the group consisting of a bulk form with filled interior, a frame form having a cavity, and a protruded form.

4. The biodegradable sensor of claim 3, wherein the bulk form with filled interior is provided as a thin film having a thickness of 50 nm to 200 μm.

5. The biodegradable sensor of claim 3, wherein the frame form having a cavity includes a dielectric frame having: a first surface facing the first electrode; a second surface spaced apart from the first surface with the cavity interposed therebetween and facing the second electrode; and a third surface connecting edges of the first and second surfaces, wherein the dielectric frame has a thickness of 10 μm to 150 μm, and a spacing between the first and second surfaces of 50 μm to 200 μm.

6. The biodegradable sensor of claim 3, wherein the protruded form of the dielectric portion includes circular or polygonal protrusions on a bottom surface, the protrusions being spaced at regular intervals.

7. The biodegradable sensor of claim 6, wherein the spacing between the bottom surfaces of the protrusions is 10 μm to 500 μm.

8. The biodegradable sensor of claim 6, wherein the height of the dielectric portion is 100 μm to 500 μm.

9. The biodegradable sensor of claim 1, wherein the dielectric constant of the dielectric portion is 0.1 pF/m to 0.5 pF/m.

10. The biodegradable sensor of claim 1, wherein the first electrode comprises at least one selected from the group consisting of magnesium (Mg), zinc (Zn), tungsten (W), and molybdenum (Mo), and the second electrode comprises at least one selected from the group consisting of magnesium (Mg), zinc (Zn), tungsten (W), and molybdenum (Mo).

11. The biodegradable sensor of claim 1, wherein the polymer comprises at least one selected from the group consisting of poly(lactic-co-glycolic acid) (PLGA), poly(buthanedithiol pentenoic anhydride) (PBTPA), polyglycolic acid (PGA), polycaprolactone (PCL), poly(glycerol sebacate) (PGS), polylactic acid (PLA), gelatin methacryloyl (GeIMA), Gelatin methacryloyl-Poly(ethylene glycol) diacrylate (GeIMA-PEGDA), and polyurethane (PU).

12. The biodegradable sensor of claim 11, wherein the polymer dielectric is PBTPA and comprises PLGA, PBTPA, and PGA in a molar ratio of 1:1:2.5 to 1:4:7.

13. A method of manufacturing a biodegradable sensor for pressure measurement according to claim 1, the method comprising: forming the first electrode comprising the first metal by applying a paste comprising the first metal or attaching a film comprising the first metal; forming a dielectric portion comprising a polymer dielectric on the first electrode; and forming the second electrode comprising the second metal on the dielectric portion by applying a paste comprising the second metal or attaching a film comprising the second metal.

14. The method of claim 13, wherein the dielectric portion is a bulk form with filled interior, and the dielectric portion is formed by screen printing or molding the polymer dielectric.

15. The method of claim 13, wherein the dielectric portion is a frame form having a cavity, and the forming of the dielectric portion comprises: forming a first surface facing the first electrode by applying the polymer dielectric; forming a third surface by applying the polymer dielectric to each edge of the first surface to define a cavity; and forming a second surface facing the second electrode on the third surface using the polymer dielectric.

16. The method of claim 13, wherein the dielectric portion is a protruded form, and the forming of the dielectric portion comprises molding the polymer dielectric into the protruded form and disposing it on the first electrode.

17. The method of claim 13, wherein the polymer dielectric is PBTPA and comprises pentenoic anhydride, triallyl triazinetrione, and butanedithiol in a ratio of 1:1:2.5 to 1:4:7.

18. The method of claim 13, further comprising repeating the steps of forming the dielectric portion and forming the second electrode after forming the second electrode to construct a multilayer structure.

19. A composite sensor assembly, comprising: a sensing unit comprising a biodegradable sensor for pressure measurement according to claim 1, wherein an electric signal changes according to an external pressure variation; and a reference unit comprising a third electrode and configured to measure a constant electric signal regardless of the external pressure variation, wherein the external pressure variation is detected using the electric signals measured from the sensing unit and the reference unit.

20. The composite sensor assembly of claim 19, wherein the sensing unit comprises the dielectric portion in a frame form with a cavity or a protruded form, and the reference unit comprises the dielectric portion in a bulk form with filled interior or has no dielectric portion.

21. The composite sensor assembly of claim 19, wherein the detection of the external pressure variation is represented by Equation 1:
$$Δ {C}_{M} = ( {ε}_{1} \frac{A}{Δ {d}_{1}} - {Δε}_{1} \frac{A}{d} ) - ( {ε}_{2} \frac{A}{Δ {d}_{2}} - {Δε}_{2} \frac{A}{d} ) \qquad [ Equation 1 ]$$
wherein ε1 is the dielectric constant of the sensing unit, A is the area of the first electrode of the sensing unit and the second electrode of the reference unit, Δd1 is the variation in spacing between the first and second electrodes in the sensing unit, Δε1 is the variation in dielectric constant due to noise in the sensing unit, d is the distance between the first and second electrodes in the sensing unit and between the third and second electrodes in the reference unit, ε2 is the dielectric constant of the reference unit, Δd2 is the variation in spacing between the third and second electrodes in the reference unit, and ΔE2 is the variation in dielectric constant due to noise in the reference unit.

22. The composite sensor assembly of claim 19, wherein the sensing unit and the reference unit are repeatedly stacked one or more times.

23. The biodegradable sensor for pressure measurement according to claim 1 is applied to a medical implant.
